(19) United States
(12) Patent Application Publication
(10) Pub. No.: US 2025/0239339 A1
(43) Pub. Date: Jul. 24, 2025

(54) App for Sharing Information About STD Status

(71) Applicant: Stacy Tru, Pacific Palisades, CA (US)

(72) Inventor: Stacy Tru, Pacific Palisades, CA (US)

(21) Appl. No.: 19/088,712

(22) Filed: Mar. 24, 2025

Related U.S. Application Data
(60) Provisional application No. 63/624,157, filed on Jan. 23, 2024.

(51) Int. Cl. G16H 10/60; G06F 21/32; G16H 80/00

(52) U.S. Cl. CPC G16H 10/60; G06F 21/32; G16H 80/00

(57) ABSTRACT

The present invention is an application to facilitate the secure sharing of STD status information among users. The app allows users to upload and share their STD test results, which can be obtained through at-home testing kits or participating clinics. The app features GPS integration to locate nearby testing facilities, a HIPAA-compliant portal for secure data handling, and encryption to protect sensitive information. Additionally, the app includes AI technology to guide users through the testing process, biometric authentication for secure access, and multi-factor authentication for added security. Users can order testing kits, view test results, and share verified STD status with potential partners while maintaining privacy and security.

The current application claims a priority to the U.S. Provisional Patent application Ser. No. 63/624,157 filed on Jan. 23, 2024.

### FIELD OF THE INVENTION

The present invention relates generally to sexual health and safety apps. More specifically, the present invention is an information sharing app that allows users to share information regarding their STD status with a current or potential sexual partner.

### BACKGROUND OF THE INVENTION

Engaging in sexual activities without knowing the STD status of the partner can make one experience shame, fear, and anxiety. With access to this knowledge beforehand, potentially life-threatening consequences can be avoided. It is an object of the present invention to provide everyone with the ability to manage their own fate—an opportunity which will forever eliminate any blurriness in matters of personal safety and well-being.

### BRIEF DESCRIPTION OF THE DRAWINGS

FIG. 1 is a flow diagram depicting how the present invention works; and

FIG. 2 shows the profile, chat, and security features of the present invention.

### DETAILED DESCRIPTION OF THE INVENTION

All illustrations of the drawings are for the purpose of describing selected versions of the present invention and are not intended to limit the scope of the present invention. As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims.

The present disclosure includes many aspects and features. Moreover, while many aspects and features relate to, and are described in the context of an app for sharing information about STD status, embodiments of the present disclosure are not limited to use only in this context.

The present invention is an app for sharing information about STD status. The present invention is an information sharing app that will be the go-to app when users need to know the STD status of current or potential sexual partner.

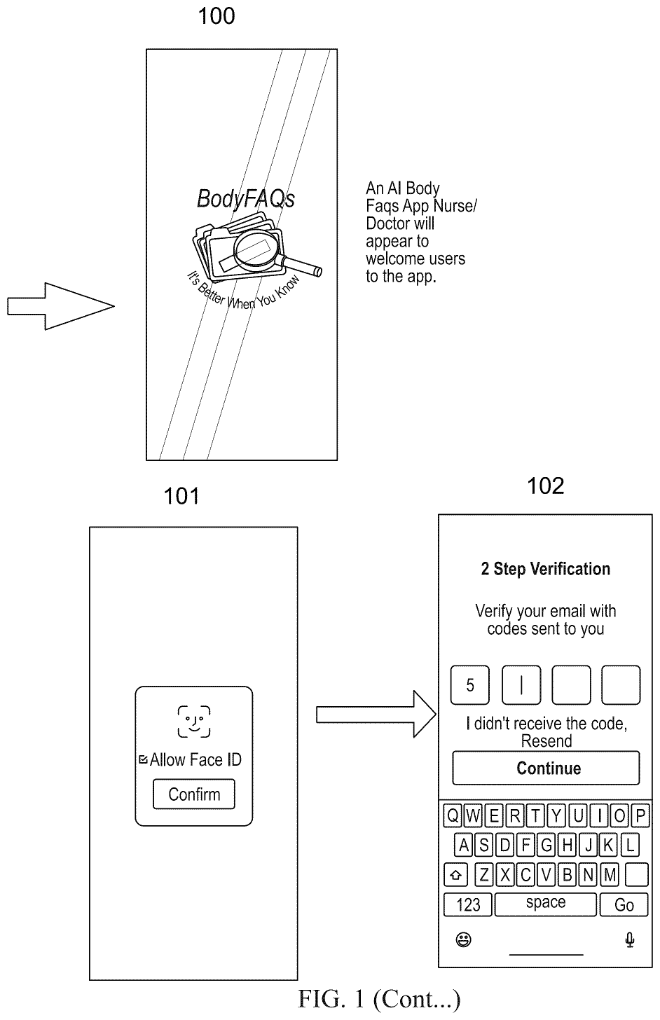

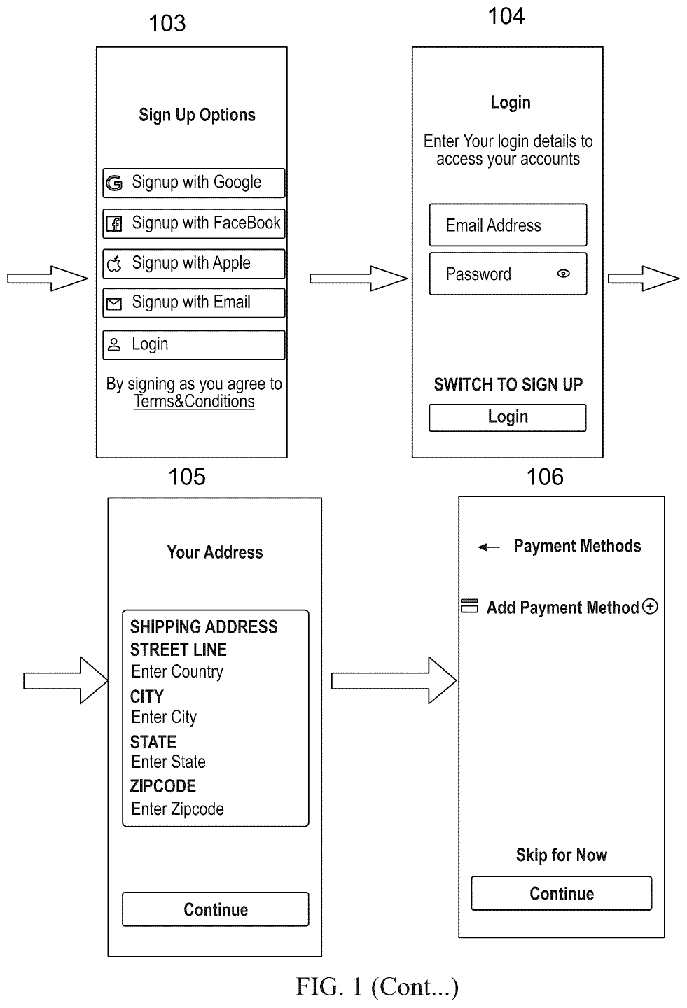

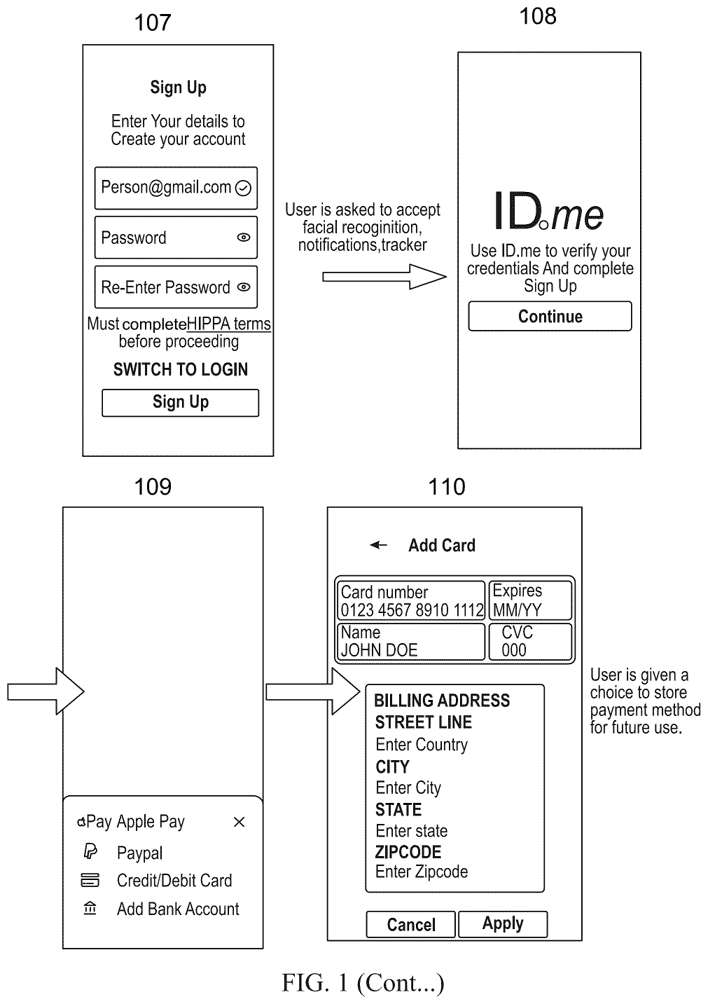

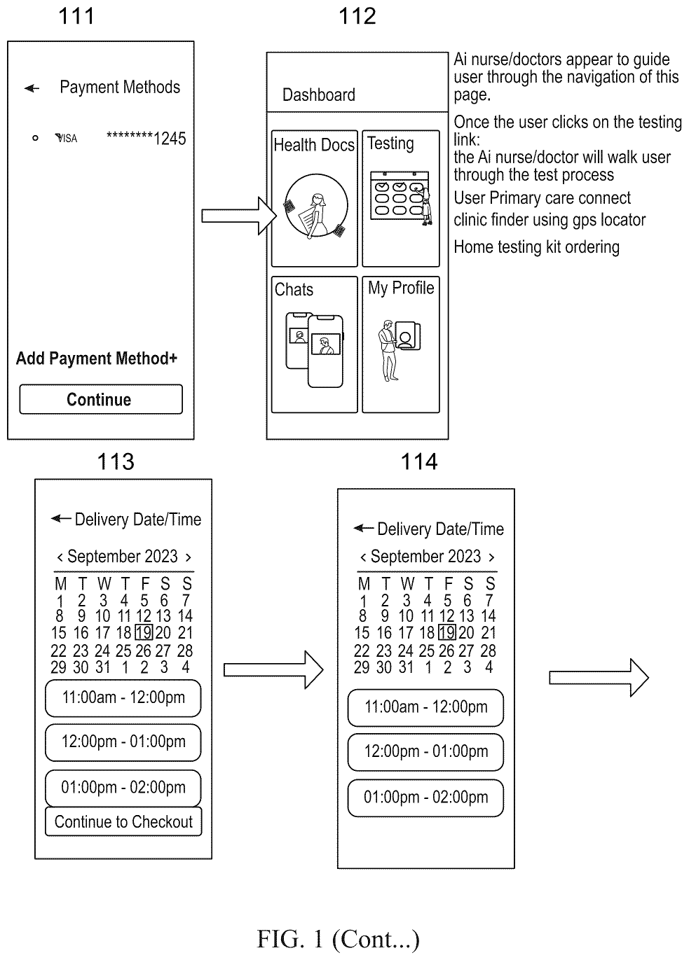

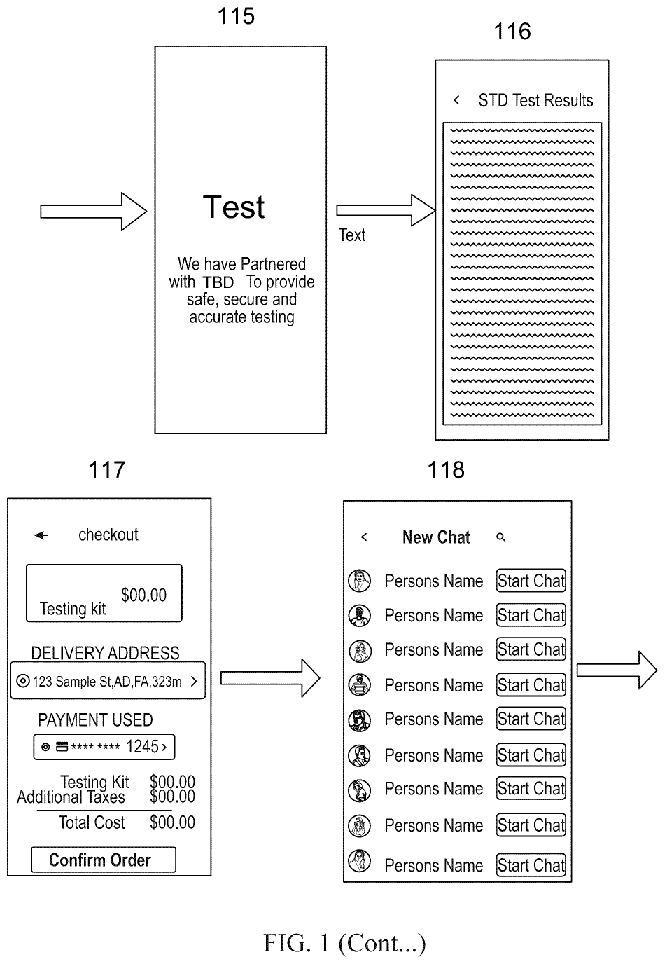

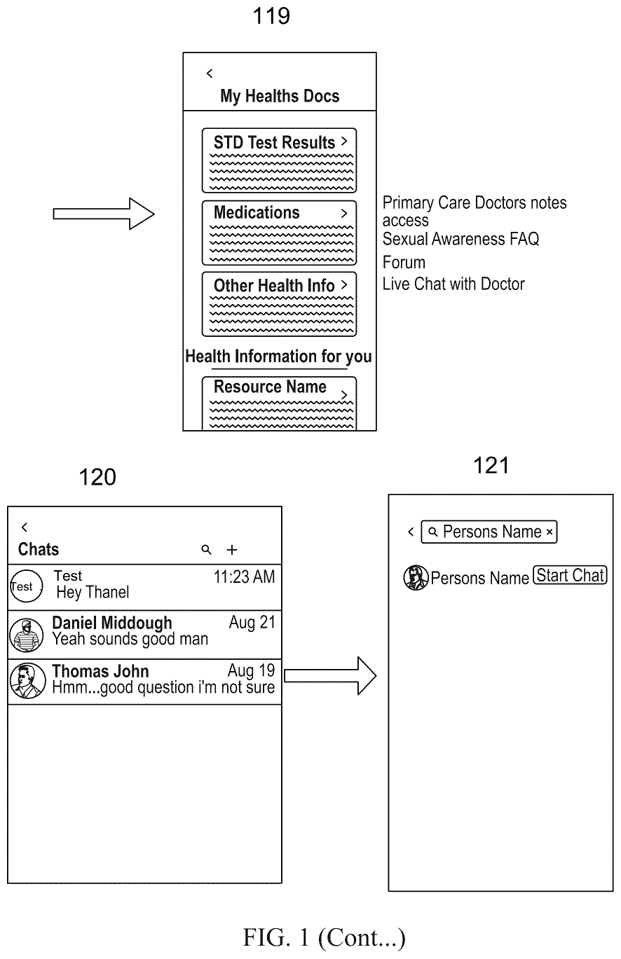

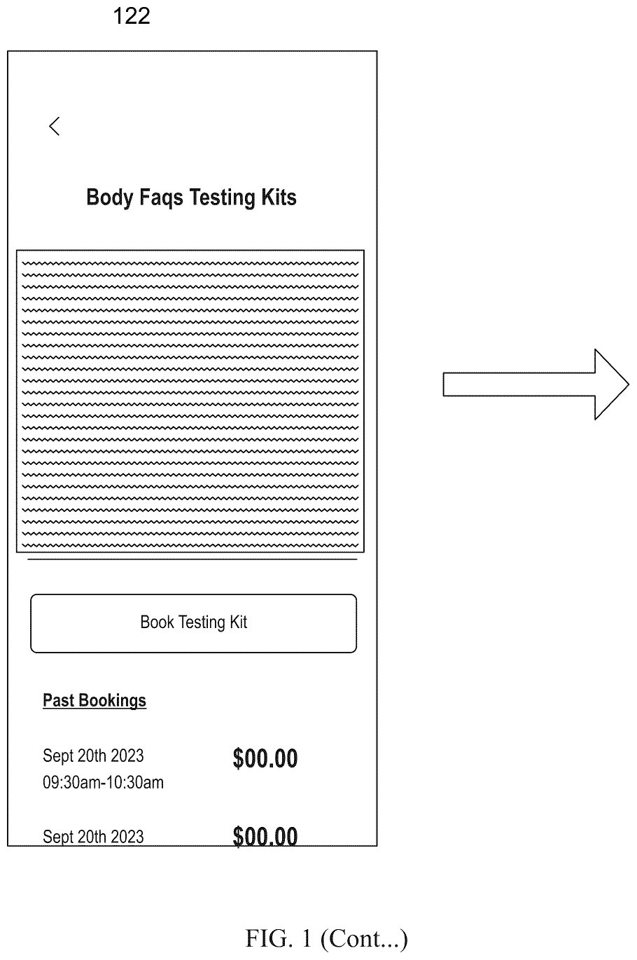

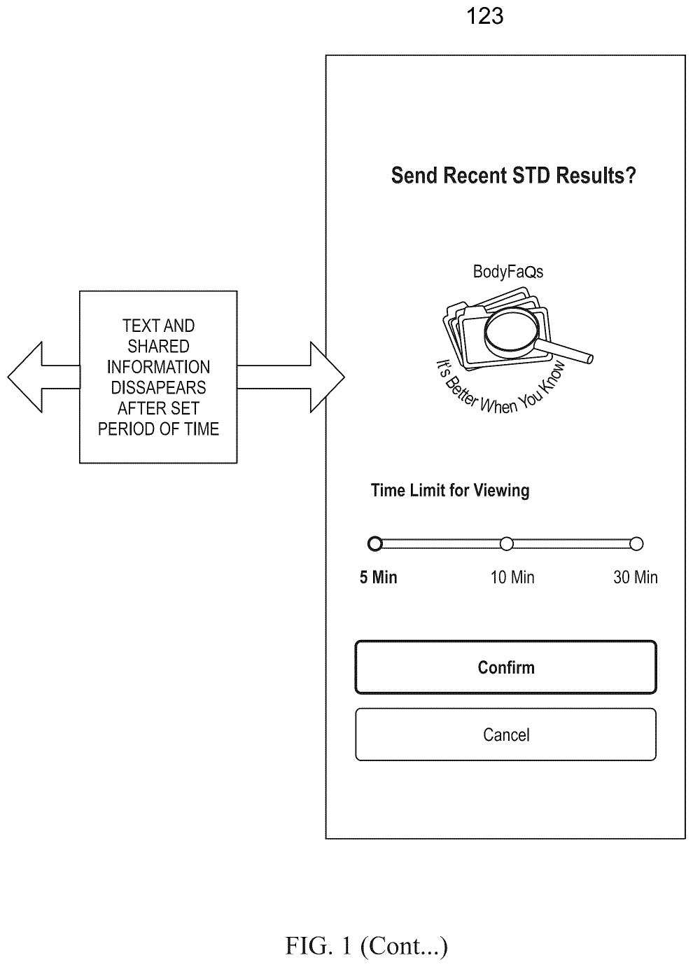

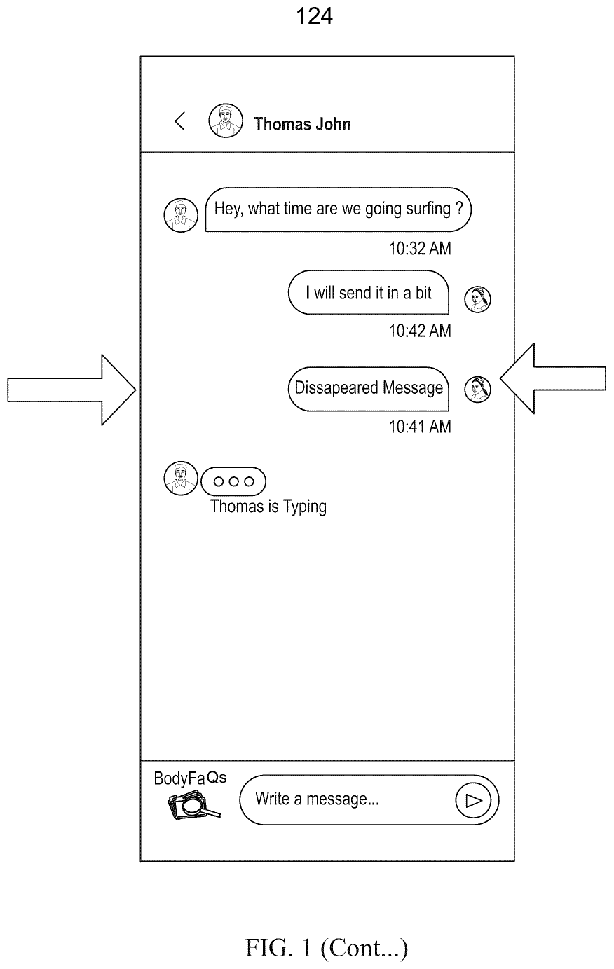

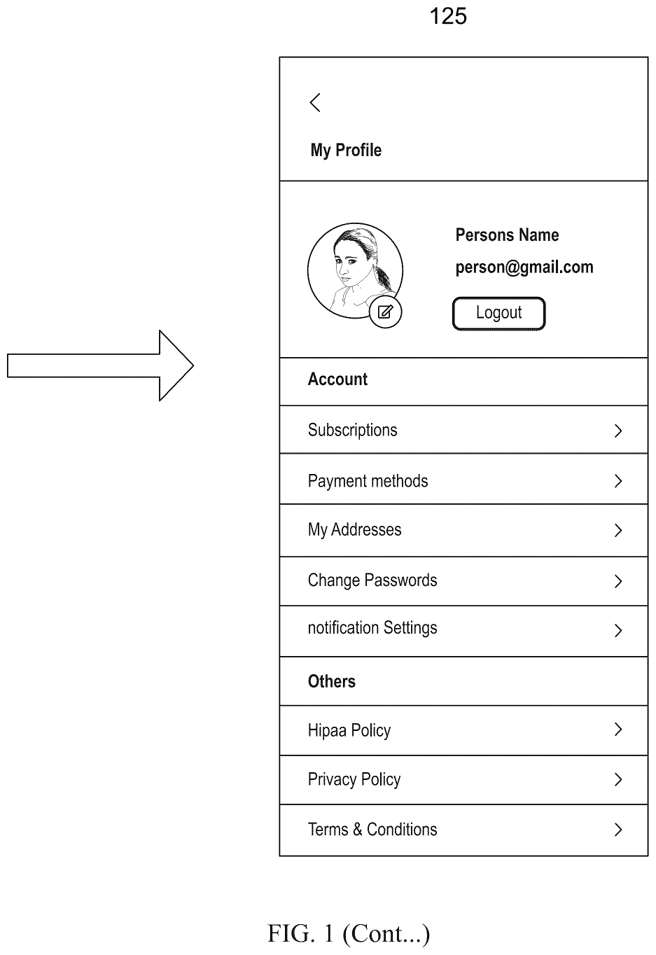

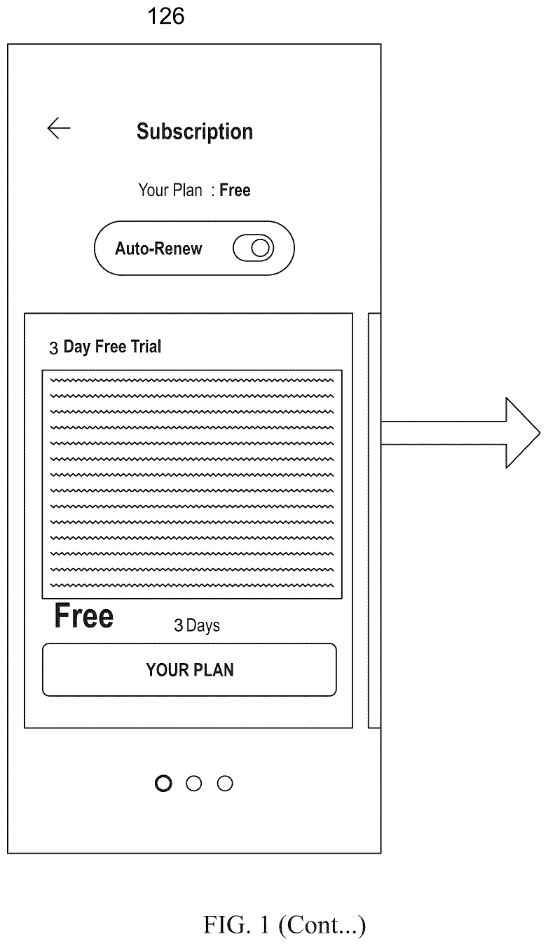

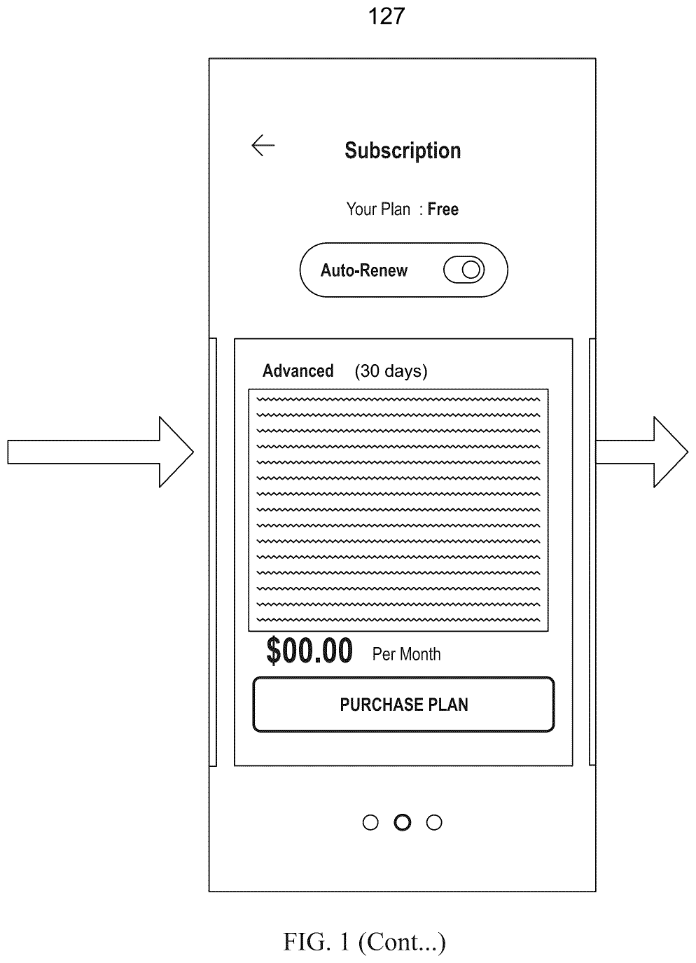

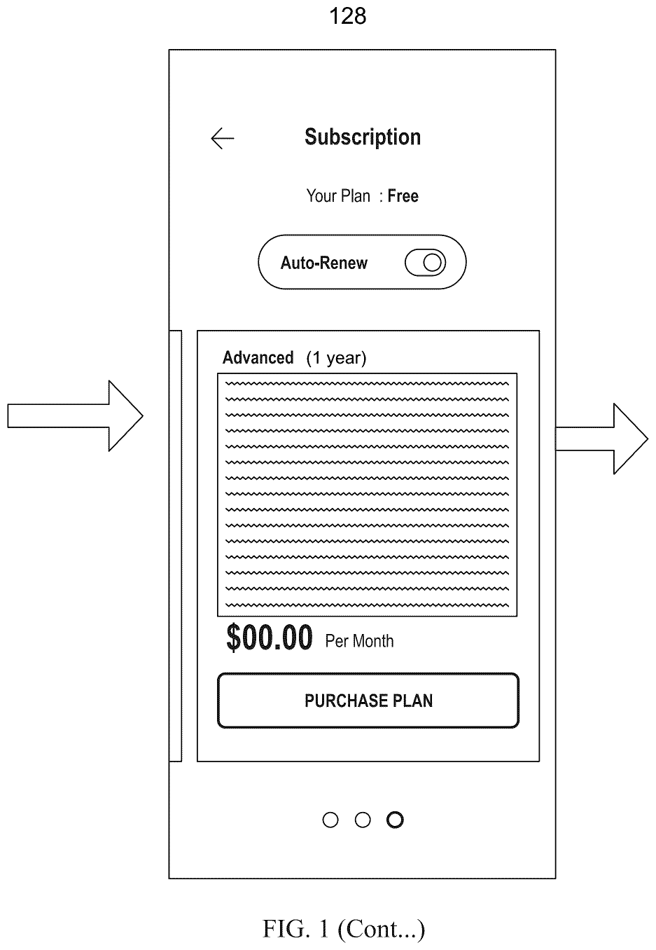

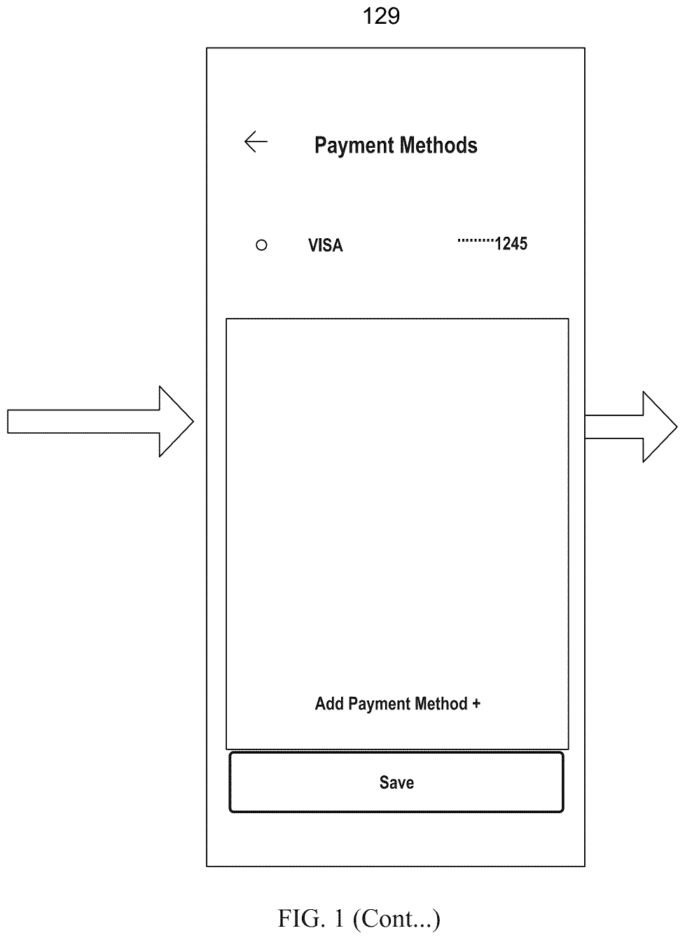

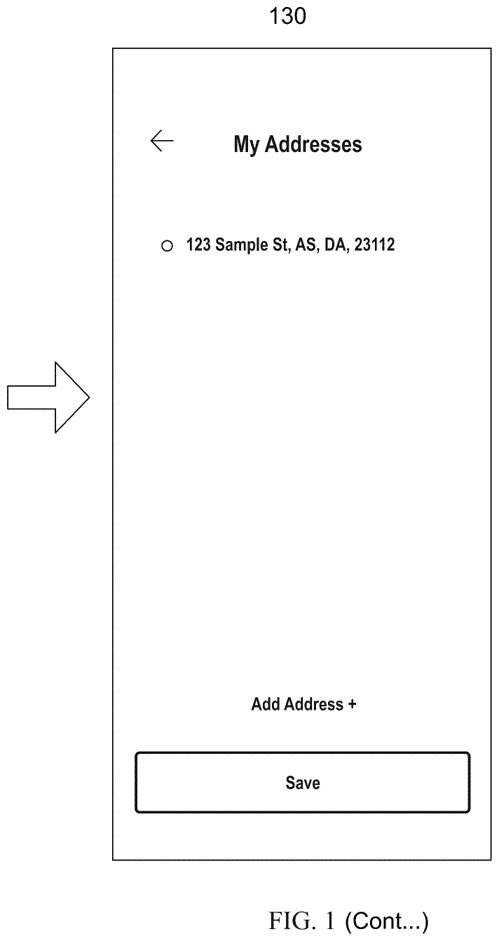

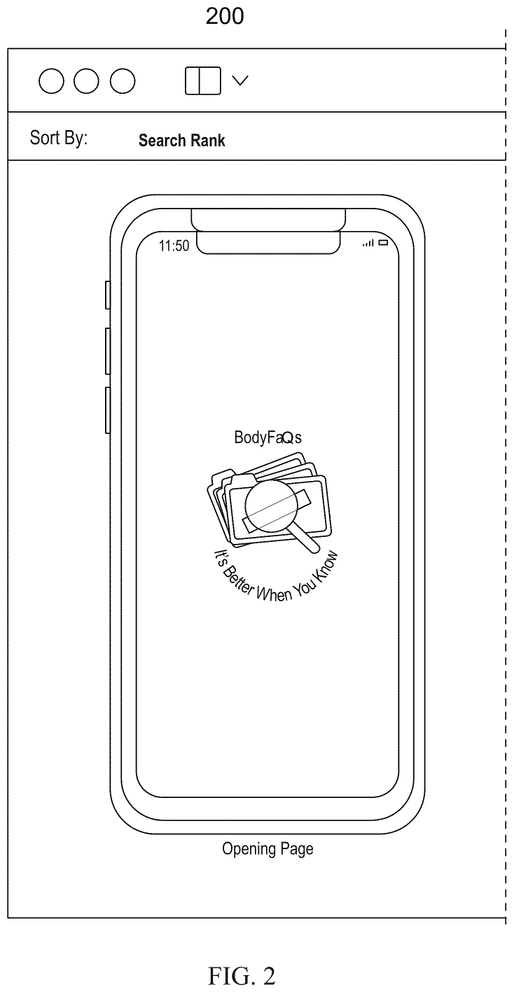

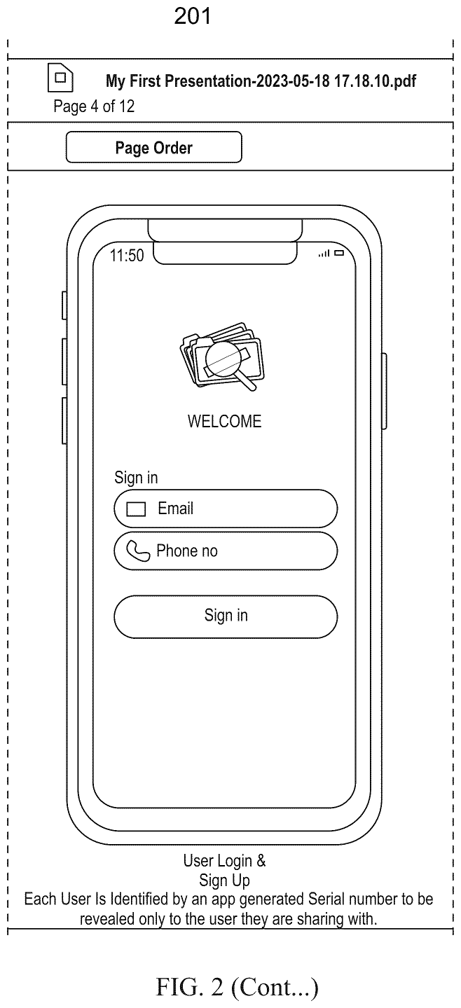

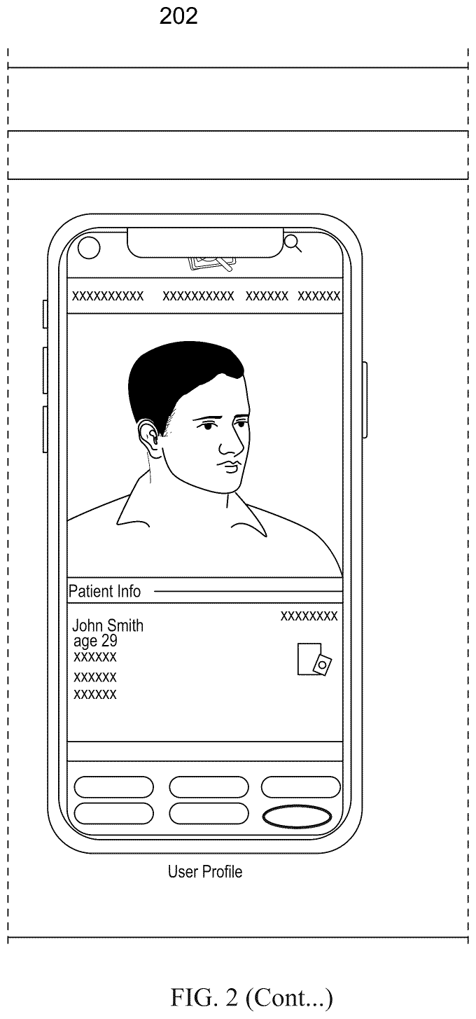

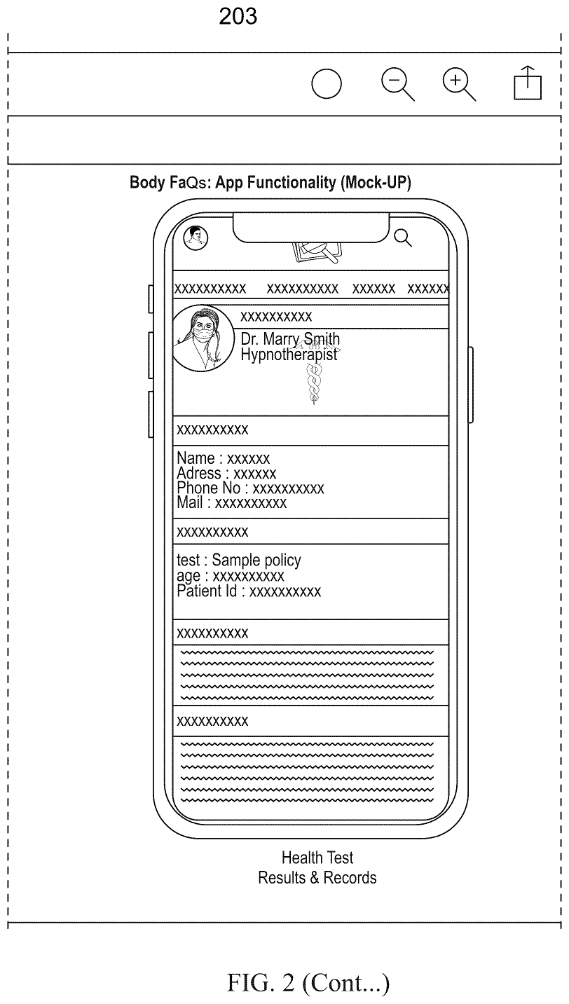

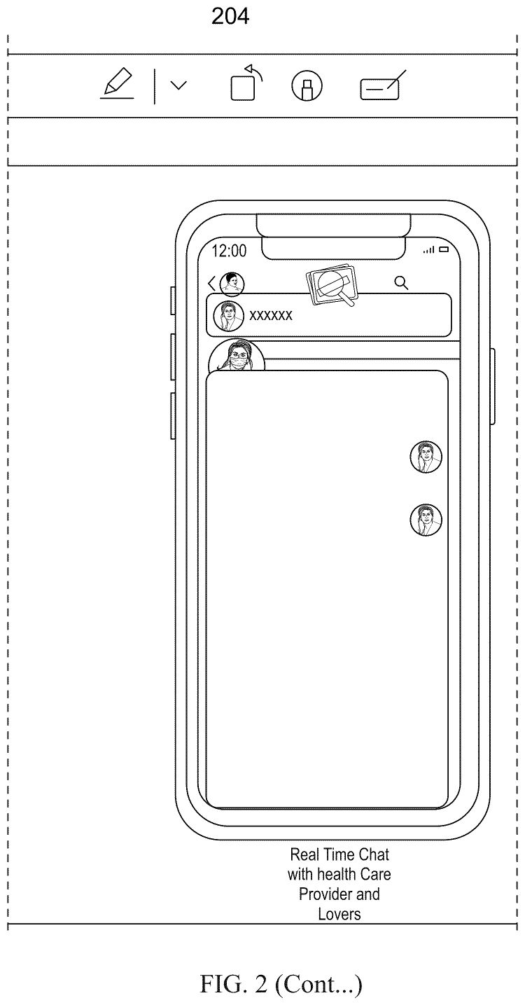

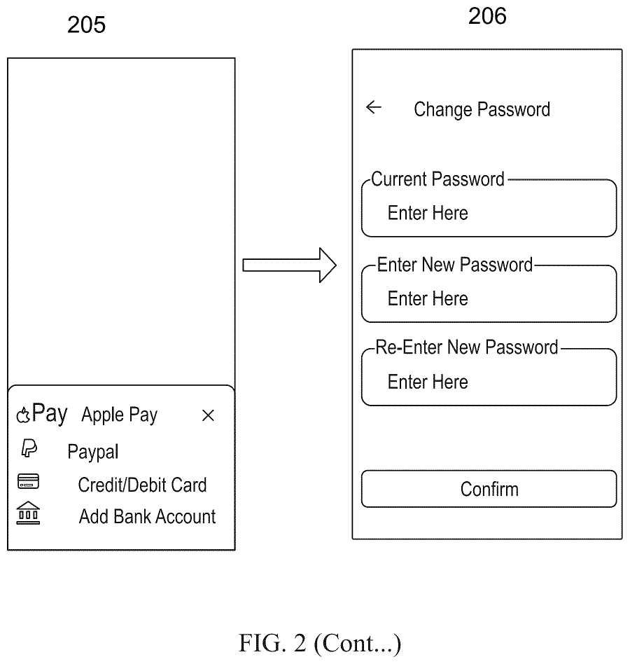

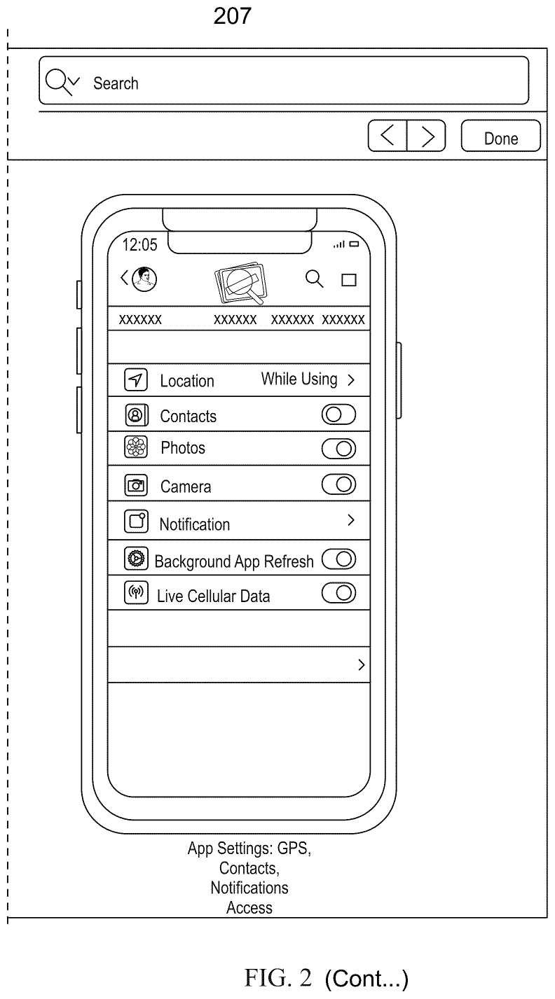

FIGS. 1 and 2 show the flow of the present invention with the placement of elements of the full functionality. It shows the present invention's opening, internal, and app end or user exit.

The present invention comprises the combination pertaining to the following software that, as a whole, creates the full functionality of the invention. Additional features may also be included.

The present invention will allow users to share sensitive STD/STI information with other app users. Additionally, tests are taken at home and at a participating clinic with results being uploaded to the app instantly upon receipt from testing kits and/or a doctor. GPS integration allows for the location for a testing facility near the user. The present invention further comprises a HIPAA high-tech compliant portal that will be adaptive to every file and include encryption and auto destruct after a set period of time. A 365 defender portal or similar portal allows for anti-file downloading from the app. Further, the present invention comprises encrypted media, extensions, and digital rates management to prevent screenshots. At-home testing kits may be order on the app and delivered to users. This is an automated process through the app to receive test results, as well as mail in results. All units will be facilitated and orchestrated through the users' primary doctor and the present invention.

The present invention further comprises AI technology (virtual nurse/doctor) to introduce the user to the present invention once they log in to the app and have landed on the home page. The AI nurse or doctor will give users a walk through the testing process, guiding the user through every step of test taking. With the use of the camera on a phone device and/or a computer, the virtual nurse may monitor the testing process of the user. Each testing process is timed and evaluated.

The present invention uses biometrics such as finger and facial recognition. Users may only access the testing platform through the identification platform and the biometrics associated within the app as follows:
Biometrics allows devices to use physical attributes—fingerprints, face, voice, or even iris color—to authenticate identity. This adds to the overall online experience, in place of passwords that are hard to remember.
Biometrics can also be used as part of the multi-factor authentication (MFA) process—especially when signing into sensitive or confidential accounts.

The use of biometrics also has widespread application in the field of healthcare. Biometric information—obtained through fingerprint and iris scanning, and facial recognition may enable hospitals to identify patients and retrieve their medical history. This ensures that healthcare facilities can provide the right treatment by having access to the correct information the users are providing and receiving.

Referring now to the figures, FIG. 1 is a flowchart detailing the process and various screens a user may encounter when using the app of the present invention. All screens are merely examples of the interface, but the screens may be organized in various ways and their appearance may vary while remaining within the scope of the present description. The home screen 100 appears when the user opens the app. A virtual doctor or nurse may also greet the user. A log-in screen 102 may use biometrics, such as Face ID as shown, to securely log the user into the platform. Additionally, two-factor authentication 102 may be used to provide additional security. The user may also sign up by connecting various other accounts 103, such as Google or Facebook. Username and password logins 104 may also be used. The user may enter their location and shipping information 105 to allow the present invention to facilitate deliveries of at-home testing kits or find testing locations in their area. The user may add a payment method 106 to pay for tests within the app or make other in-app purchases. The sign-up screen 107 allows users to create their username and password and may further require agreement to confidentiality terms. The identification screen 108 allows users to set up and verify credentials. The payment screen 109 allows users to choose a payment method when making purchases. The user may enter billing information on the billing screen 110. An add payment method screen 111 allows users to view existing methods and add new methods or cards. The dashboard 112 allows users to navigate to other features of the app, such as health documents, testing information, chats, and the user profile. The delivery screens 113 and 114 allow the user to choose a date and time for delivery of a testing kit. The testing screen 115 may show the company which helps the app facilitate testing. A user may view their STD test results 116. The checkout screen 117 shows the details of a user's testing kit purchase. The chat screens 118, 120, and 121 show the user's chats with various other users. The health documents screen 119 shows various documents related to the user, such as test results, medications, and doctors' notes. The testing kit information screen 122 allows users to order testing kits and view additional related information. The user chat screen 124 is where the user can send and receive messages to and from another user. The STD status sharing screen 123 allows users to send verified STD test results to another user and set a time limit for viewing. If the user sets a time limit, the other user will no longer be able to view the results after the allotted time, ensuring that results stay private. The present invention may further comprise software or a setting that prevents another user from taking a screenshot of the shared information, further increasing the security and privacy of the app. Alternatively, or additionally, the app may send a notification to a user when another user takes a screenshot of the information they send. Additional security features are further contemplated as needed to maintain the security and privacy of the sensitive information shared within the app of the present invention. The profile page 125 shows the user's information and allows navigation to change settings and view other information about the user's account. The app of the present invention may require a subscription, and the subscription screen 126 allows users to purchase a subscription 127 or sign up for a free trial. Referring to FIG. 1, the trial period in the drawing is 3 days, but other trial periods are possible. The user may also enable auto-renew to ensure their subscription remains active. Subscription lengths may also vary, such as 30 days, 60 days, or 1 year. The 1 year subscription screen 128 is an example, but subscriptions may be for any amount of time. Further the drawings show the price as $00.00 per month, but that number is a placeholder, and subscription prices may vary. The subscriptions may further include a certain number of testing kits available to order depending on the length and level of the subscription. The user selects a payment method 129 to purchase the subscription. The address screen 130 shows the user's address information and allows the user to add a new address.

FIG. 2 shows further features of the app of the present invention. FIG. 2 shows a home screen 200 and a welcome page 201 where the user signs in to the app. The user profile 202 shows a profile picture, name, age, and other information about the user. The present invention may also include profiles for medical professionals. The doctor profile screen 203 provides the user with information about their doctor or a potential doctor. The health care provider chat 204 allows the user to chat with their doctor or nurse. The user may select other payment methods 205 for their subscription. The change password screen 206 allows the user to change their password. The user settings page 207 allows the user to view and change various settings, such as location tracking, contact synchronization, photos, cameras, notifications, background app refresh, and cellular data. Additional setting are also possible.

The present invention is an app allowing users to share STD information with other users to ensure safe sexual experiences. The app ensures privacy and security due to the sensitivity of the information being shared. The present invention makes it easy for people to verify their STD status and share it with potential sexual partners.

Although the invention has been explained in relation to its preferred embodiment, it is to be understood that many other possible modifications and variations can be made without departing from the spirit and scope of the invention.

What is claimed is:

1. A system for sharing STD status information, comprising;
an application interface for user interaction;
means for uploading and sharing STD test results obtained from at-home testing kits or participating clinics;
GPS integration for locating nearby testing facilities;
a HIPAA-compliant portal for secure data handling;
encryption mechanisms to protect sensitive information;
AI technology to guide users through the testing process;
biometric authentication for secure access; and
multi-factor authentication for added security.

2. The system of claim 1, wherein the application interface comprises;
a home screen displaying a virtual doctor or nurse;
a log-in screen utilizing biometric authentication and two-factor authentication; and
screens for entering location, shipping information, and payment methods;
a dashboard for navigating health documents, testing information, chats, and user profiles.

3. The system of claim 1, further comprising;
means for ordering at-home testing kits through the application; and
automated processes for receiving and uploading test results from testing kits or clinics.

4. The system of claim 1, wherein the AI technology comprises;
a virtual nurse or doctor providing a walkthrough of the testing process; and
monitoring of the testing process using the device's camera.

5. The system of claim 1, wherein the biometric authentication comprises;
fingerprint recognition; and
facial recognition.

6. The system of claim 1, further comprising;
a means for sharing verified STD test results with other users with an option to set a time limit for viewing.

7. The system of claim 1, wherein the HIPAA-compliant portal comprises;
adaptive file handling with encryption and auto-destruct features after a set period; and
anti-file downloading mechanisms.

8. The system of claim 1, further comprising;
encrypted media extensions and digital rights management to prevent screenshots.

9. The system of claim 1, wherein the application interface further comprises;
subscription management screens allowing users to purchase subscriptions or sign up for free trials;
user settings screens for managing location tracking, contact synchronization, photos, cameras, notifications, background app refresh, and cellular data.

10. The system of claim 1, further comprising;
a means for facilitating communication between users and healthcare providers through chat interfaces.
